(19) United States
(12) Patent Application Publication
(10) Pub. No.: US 2005/0154327 A1
(43) Pub. Date: Jul. 14, 2005

(54) Thermometer

(76) Inventor: Tsutomu Nakazawa, Isesaki-shi (JP)

Correspondence Address: WATCHSTONE P + D, 1300 EYE STREET, NW, SUITE 400 EAST, WASHINGTON, DC, 20005, US

(21) Appl. No.: 10/983,093

(22) Filed: Nov. 8, 2004

Related U.S. Application Data
(63) Continuation-in-part of application No. 10/948,750, filed on Sep. 24, 2004.
Application No. 10/948,750 is a continuation-in-part of application No. PCT/JP03/03437, filed on Mar. 20, 2003.

(30) Foreign Application Priority Data
Mar. 20, 2002 (JP) 2002-78049
Nov. 6, 2003 (JP) 2003-377127

(51) Int. Cl. A61B 5/00; G01K 7/00

(52) U.S. Cl. 600/549; 128/903; 374/100

(57) ABSTRACT

In some embodiments, a thermometer is disclosed that can be used in combination with a reader for reading temperature information of a patient from the thermometer pad. In some examples, the thermometer includes, e.g., a thermal sensor that changes in resistance in response to temperature changes, a memory, and a temperature correction circuit. The memory stores, in advance, a temperature difference between an expected measured temperature as a true temperature and an actually measured temperature corresponding to the expected measured temperature obtained by converting an analog signal from the thermal sensor into a digital signal as a correction value. The temperature correction circuit corrects a temperature measured in an actual use with the correction value.

### CROSS REFERENCE TO RELATED APPLICATIONS

This is continuation-in-part of commonly assigned co-pending U.S. patent application Ser. No. 10/948,750 (Attorney Docket No. 2905-116) filed on Sep. 24, 2004, which is a continuation-in-part of commonly assigned co-pending PCT application No. PCT/JP03/03437, filed on Mar. 20, 2003, designating the United States of America as one of designation countries and claiming the benefit of the filing date of Japanese Patent Application No. 2002-78049 filed on Mar. 20, 2002, the entire disclosures of which are incorporated herein by reference in their entireties.

This application also claims priority under 35 U.S.C. §119 to Japanese Patent Application No. P2003-377127 filed on Nov. 6, 2003, the entire disclosure of which is incorporated herein by reference in is entirety.

### BACKGROUND OF THE INVENTION

1. Field of the Invention

The preferred embodiments of the present invention relate, inter alia to an attachable clinical thermometer and/or a temperature measuring pad to be used in connection with a reader for reading temperature information of a patient from the thermometer or the measuring pad.

2. Description of the Related Art

The following description sets forth the inventor's knowledge of related art and problems therein and should not be construed as an admission of knowledge in the prior art.

In hospitals, by way of example, it is necessary for a nurse to measure body temperatures of patients to monitor their health status several times a day. In measuring the body temperatures, conventionally, mercury thermometers and/or electric thermometers have been generally used. Typically, such thermometers are delivered to respective patients to measure their body temperature. Often, the patients measure their respective body temperatures by themselves. Then, in common scenarios, a nurse reads respective body temperatures of the thermometers and writes down the measured values on a recording sheet and collects the thermometers. Thereafter, it is often necessary for a nurse to sterilize the collected thermometers and then input the measured results which were once written down on the sheet into a personal computer.

Typically, there were, among other problems, the following drawbacks in measuring patient's body temperatures in hospitals.

In most hospitals, in order to check the health condition of each patient, such as, e.g., that the body temperature of each patient will be measured at least three times a day, e.g., once in the morning, once at noon and once at night in the case of using mercury thermometers, it takes a long time to complete the measurement. On the other hand, in the case of using prediction type electric thermometers, it is required to tightly fit the thermometer on a skin surface to obtain the equilibrium body temperature. Otherwise, the accuracy deteriorates.

The measured temperatures of all of the patients is recorded on a recording sheet with a pencil or the like together with necessary information connected with the measured temperatures, such as, e.g., the patient's name and the measured date and time. Therefore, a nurse is required to complete the recoding operation in addition to the body temperature measuring operation, causing troublesome operations. Furthermore, as mentioned above, the nurse is further required to input the measured data into a computer using a keyboard, which further increases the burden of the nurse. On the other hand, for each patient, the body temperature measuring operation was also troublesome.

In order to overcome the above mentioned and/or other drawbacks, the present inventor has proposed, inter alia, a unique attachable thermometer and a system using the same in his previously filed application. In some of the preferred embodiments thereof, a thermistor can be used as a temperature sensor. However, there are additional areas that may be improved upon. In manufacturing thermistors, thermistors with different characteristics can be obtained, which may affect the accuracy of measuring temperature. On the other hand, even if thermistors that are equal in characteristic could be manufactured, it has been too difficult to manufacture attachable thermometers with a built-in thermistor such that all or substantially all of the thermometers have temperature measuring accuracies falling within a predetermined accuracy range due to assembling variations and other factors. Accordingly, tight selection tests and/or processing were required, thereby resulting in further demands to avoid an increased number of defective goods, which in turn increases the manufacturing cost. Under the circumstances, it was potentially difficult to provide an attachable thermometer with high accuracy at low cost.

The description herein of potential advantages and/or disadvantages of various features, embodiments, methods and apparatus disclosed in other documents is in no way intended to limit the various embodiments of the present invention, nor to limit, in any way, the broadest scope of protection afforded by the present assignee's above-noted prior applications. For example, certain features of the preferred embodiments of the invention may be capable of overcoming certain disadvantages and/or providing certain advantages, such as, e.g., potential disadvantages and/or advantages discussed herein, while retaining some or all of the features, embodiments, methods, and apparatus disclosed therein.

### SUMMARY OF THE INVENTION

The preferred embodiments of the present invention have been developed in view of the above-mentioned and/or other problems in the related art. The preferred embodiments of the present invention can significantly improve upon existing methods and/or apparatuses.

Among other potential advantages, some embodiments can provide an adhesive clinical thermometer pad capable of measuring a body temperature of a patient in hospitals or the like with high accuracy.

Among other potential advantages, some embodiments can provide a temperature measuring pad with high accuracy at low cost.

According to some embodiments of the present invention, a thermometer includes:
a thermal sensor that changes in resistance in response to temperature changes;
a memory; and
a temperature correction circuit,
wherein the memory stores, in advance, a temperature difference between an expected measured temperature as a true temperature and an actually measured temperature corresponding to the expected measured temperature obtained by converting an analog signal from the thermal sensor into a digital signal as a correction value, and
wherein the temperature correction circuit corrects a temperature measured in an actual use with the correction value.

According to some embodiments of the present invention, in a thermometer to be used in combination with a reader for reading temperature information from the thermometer, the thermometer includes:
an antenna portion for receiving a radio wave emitted from a reader;
an electric power generating portion for generating electric power with the radio wave received by the antenna portion;
a thermal sensor that changes in resistance in response to temperature changes;
an A/D converter for converting an analog signal from the thermal sensor into a digital signal;
an output portion for wirelessly outputting temperature information toward the reader;
a memory; and
a temperature correction circuit,
wherein the memory stores, in advance, a temperature difference between an expected measured temperature as a true temperature and an actually measured temperature corresponding to the expected measured temperature obtained by converting an analog signal from the thermal sensor into a digital signal as a correction value,
wherein the temperature correction circuit corrects a temperature measured in an actual use with the correction value to obtain a corrected temperature, and
wherein temperature information including the corrected temperature is wirelessly outputted toward the reader from the output portion via the antenna portion.

According to some embodiments of the present invention, in a thermometer to be used in combination with a reader for reading temperature information from the thermometer, the thermometer includes:
an antenna portion for receiving a radio wave emitted from a reader;
an electric power generating portion for generating electric power with the radio wave received by the antenna portion;
a thermal sensor that changes in resistance in response to temperature changes;
an A/D converter for converting an analog signal from the thermal sensor into a digital signal;
an output portion for wirelessly outputting temperature information toward the reader;
a memory; and
a temperature correction circuit,
wherein the memory stores, in advance, at least one expected measured temperature as a true temperature and at least one actually measured temperature corresponding to the at least one expected measured temperature obtained by converting the analog signal from the thermal sensor into the digital signal,
wherein the temperature correction circuit corrects a temperature measured in an actual use with a ratio of the at least one actually measured temperature to the at least one expected measured temperature as a true temperature to obtain a corrected temperature, and
wherein temperature information including the corrected temperature is wirelessly outputted toward the reader from the output portion via the antenna portion.

According to some embodiments of the present invention, in a thermometer to be used in combination with a reader for reading temperature information from the thermometer, the thermometer includes:
an antenna portion for receiving a radio wave emitted from a reader;
an electric power generating portion for generating electric power with the radio wave received by the antenna portion;
a thermal sensor that changes in resistance in response to temperature changes;
an A/D converter for converting an analog signal from the thermal sensor into a digital signal;
an output portion for wirelessly outputting temperature information toward the reader;
a memory; and
a temperature correction circuit,
wherein the memory stores, in advance, a plurality of expected measured temperatures as true temperatures and a plurality of actually measured temperatures each corresponding to each of the plurality of expected measured temperatures obtained by converting the analog signal from the thermal sensor into the digital signal,
wherein the temperature correction circuit corrects a temperature measured in an actual use with a temperature difference between the expected measured temperature and the actually measured temperature corresponding to the temperature measured in an actual use to obtain a corrected temperature, and
wherein temperature information including the corrected temperature is wirelessly outputted toward the reader from the output portion via the antenna portion.

In some example, the temperature correction circuit can change the correction value depending on a divided temperature range corresponding to the actually measured temperature.

In some examples, the temperature correction circuit changes the correction value depending on the actually measured temperature by using a mathematical formula.

According to some embodiments of the present invention, in a thermometer to be used in combination with a reader for reading temperature information from the thermometer, the thermometer includes:
an antenna portion for receiving a radio wave emitted from a reader;
an electric power generating portion for generating electric power with the radio wave received by the antenna portion;
a thermal sensor that changes in resistance in response to temperature changes;
an A/D converter for converting an analog signal from the thermal sensor into a digital signal;
an output portion for wirelessly outputting temperature information toward the reader;
a memory;
a temperature correction circuit; and
a control circuit,
wherein the memory stores, in advance, temperature differences between a plurality of expected measured temperatures as true temperatures and a plurality of actually measured temperatures corresponding to each of the plurality of the expected measured temperatures each obtained by converting an analog signal from the thermal sensor into a digital signal as a correction value, and
wherein the control circuit controls the output portion so that the output portion transmits the temperature measured in an actual use and the correction value corresponding to the temperature measured in an actual use toward the reader from the output portion via the antenna portion.

In some examples, the thermal sensor can be a thermistor.

In some examples, the thermometer can be an adhesive thermometer pad used for measuring a body temperature of a patient for clinical purposes.

According to some embodiments of the present invention, in an adhesive clinical thermometer pad to be used in combination with a reader for reading temperature information of a patient from the thermometer pad, the adhesive clinical thermometer pad includes:
a flexible main body of a generally flat shape;
an adhesive layer formed on a rear surface of the main body;
an antenna portion for receiving a radio wave emitted from a reader;
an electric power generating portion for generating electric power with the radio wave received by the antenna portion;
a temperature sensor for measuring a body temperature of the patient; and
an output portion for wirelessly outputting temperature information toward the reader,
wherein the antenna portion, the electric power generating portion, the temperature sensor and the output portion are embedded in the main body,
wherein the temperature information includes a measured temperature and an ID code given to the clinical thermometer pad, and
wherein the output portion is operated by the electric power generated by the electric power generating portion,
whereby the adhesive clinical thermometer pad when attached to a skin surface of the patient via the adhesive layer receives a radio wave from the reader, generates electric power from the received radio wave, measures the body temperature of the patient and wirelessly outputs the temperature information toward the reader.

In some examples, the adhesive clinical thermometer pad can further include a memory for storing the ID code. Preferably, the memory is a rewritable memory, so that the ID code can be rewritten.

In some examples, the adhesive clinical thermometer pad can further include an A/D converter for converting an analog signal from the temperature sensor into a digital signal, and wherein the digital signal is wirelessly outputted from the output portion via the antenna.

In some examples, the adhesive clinical thermometer pad can be configured to be connected to a computer via the reader, whereby the computer reads the temperature information, stores the read temperature information, processes the read temperature information and displays the processed information.

According to some embodiments of the present invention, in a temperature measuring pad to be used in combination with a reader for reading temperature information from the temperature measuring pad, the temperature measuring pad including:
an antenna portion for receiving an electromagnetic wave emitted from a reader;
an electric power generating portion for generating electric power with the electromagnetic wave received by the antenna portion;
a temperature sensor for sensing the temperature of an object; and
an output portion for wirelessly outputting temperature information toward the reader, the temperature information including a sensed temperature and an ID code given to the temperature measuring pad;
wherein the output portion is operated by the electric power generated by the electric power generating portion.

In some examples, in the temperature measuring pad, the temperature measuring pad can include a flexible main body of a generally flat shape and an adhesive layer formed on a rear surface of the main body, and wherein the antenna portion, the electric generating portion, the temperature sensor and the output portion are contained upon the main body.

In some examples, the temperature measuring pad can further includes an A/D converter for converting an analog signal from the temperature sensor into a digital signal, and wherein the digital signal is wirelessly outputted from the output portion via the antenna portion.

In some examples, the temperature measuring pad can further include a memory for storing the ID code. Preferably, the memory is a rewritable memory.

In some examples, the adhesive clinical thermometer pad can be configured to be connected to a computer via the reader, whereby the computer reads the temperature information, stores the read temperature information, processes the read temperature information and displays the processed information.

In some examples, the temperature measuring pad can be used for measuring a body temperature of a patient for clinical purposes.

According to some embodiments of the present invention, in a temperature measuring pad to be used in combination with a reader for reading temperature information from the temperature measuring pad, the temperature measuring pad including:
an adhesive main body of a generally flat shape;
a power source;
a temperature sensor for sensing a temperature of an object; and
an output portion for wirelessly outputting temperature information toward the reader, the temperature information including a sensed temperature and an ID code given to the temperature measuring pad;
wherein the power source, the temperature sensor and the output portion are contained upon the main body, and
wherein the output portion is operated by the power source.

In some examples, the adhesive main body can have an adhesive layer on a rear surface thereof, and wherein the battery, the temperature sensor and the output portion are embedded in the main body.

In some examples, the temperature measuring pad can further include an A/D converter for converting an analog signal from the temperature sensor into a digital signal, and wherein the digital signal is wirelessly outputted from the output portion.

In some examples, the temperature measuring pad can include a memory for storing the ID code. Preferably, the memory is a rewritable memory.

In some examples, the temperature measuring pad can be configured to be connected to a personal computer via the reader, whereby the personal computer reads the temperature information, stores the read temperature information, processes the read temperature information and displays the processed information.

In some examples, the temperature measuring pad can be used for measuring a body temperature of a patient for clinical purposes.

The above and/or other aspects, features and/or advantages of various embodiments will be further appreciated in view of the following description in conjunction with the accompanying figures. Various embodiments can Include and/or exclude different aspects, features and/or advantages where applicable. In addition, various embodiments can combine one or more aspect or feature of other embodiments where applicable. The descriptions of aspects, features and/or advantages of particular embodiments should not be construed as limiting other embodiments or the claims.

### BRIEF DESCRIPTION OF THE DRAWINGS

The preferred embodiments of the present invention are shown by way of example, and not limitation, in the accompanying figures, in which:

FIG. 1A is a top view of a temperature measuring pad according to an embodiment of the present invention;

FIG. 1B Is a cross-sectional view taken along the line 1-1 in FIG. 1A;

FIG. 2 is a block diagram of the pad;

FIG. 3 shows an illustrative comprehensive temperature measuring system related to some embodiments of the present invention;

FIG. 4 is a block diagram of a reader (receiving portion) and that of an adhesive temperature measuring pad (transmitting portion) of the temperature measuring system;

FIG. 5 is a flowchart of the operation of the system;

FIG. 6 is a block diagram of the reader (receiving portion) and that of a computer connected to the reader;

FIG. 7 is an example of data stored in the computer;

FIG. 8A is an organized data displayed on a screen of the computer;

FIG. 8B is a graph of the organized data displayed on the screen of the computer;

FIG. 9 shows a state in which an ID code of the adhesive temperature pad is being rewritten;

FIG. 10 is a block diagram of the system shown in FIG. 9;

FIG. 11 shows an entire view showing test processing of a thermometer;

FIG. 12 is an example of a graph showing test results;

FIG. 13 is a table showing the test results;

FIG. 14 is another example of a graph showing test results;

FIG. 15 is a table showing the test results;

FIG. 16 is a table showing correction values; and

FIG. 17 is a still another example of a graph showing test results.

### DETAILED DESCRIPTION OF THE PREFERRED EMBODIMENTS

In the following paragraphs, some preferred embodiments of the invention will be described by way of example and not limitation. It should be understood based on this disclosure that various other modifications can be made by those in the art based on these illustrated embodiments.

A preferable embodiment of the present invention will be explained with reference to the attached drawings. The following explanation will be directed to, inter al, an adhesive clinical thermometer pad used for measuring body temperatures of patients in hospitals. However, it should be understood that the present invention is not limited to the above and can also be applied to various applications required to measure a surface temperature of an object in various industries.

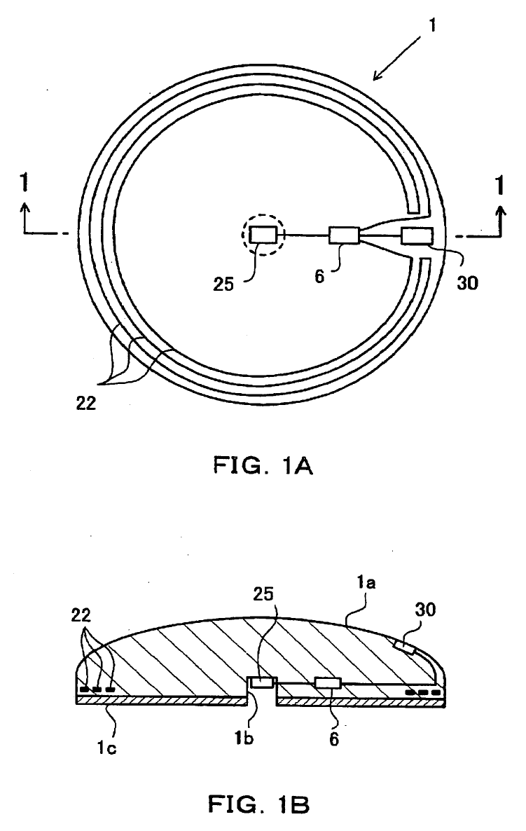

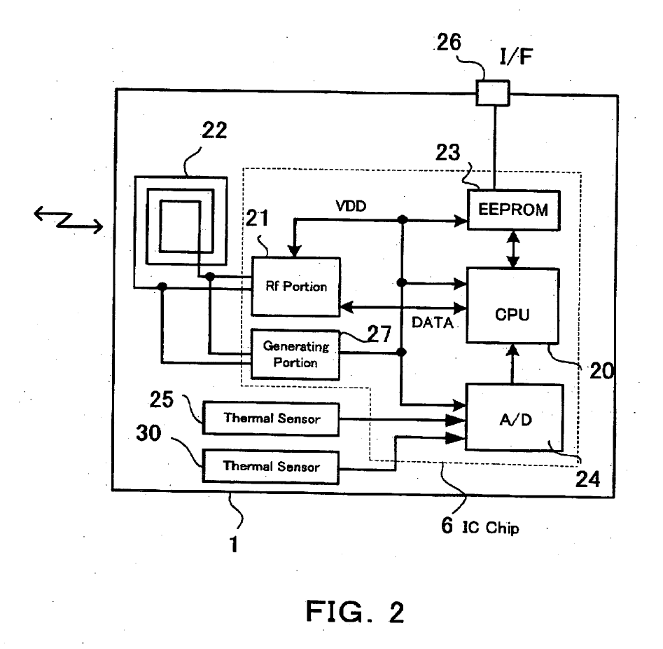

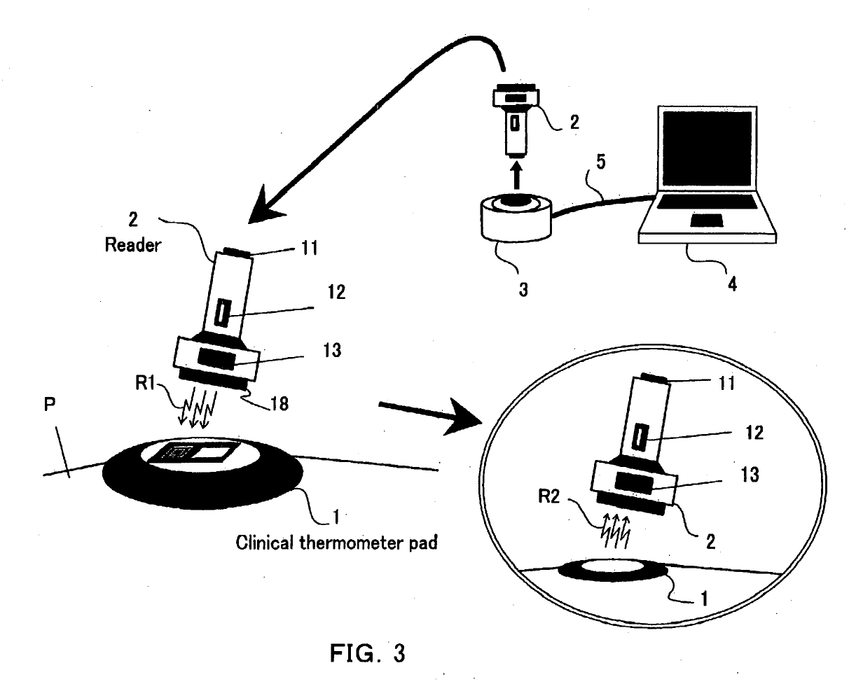

FIG. 1 is a schematic view showing an illustrative adhesive clinical thermometer pad (temperature measuring pad) of the preferred embodiments. A top view of the pad 1 is shown in FIG. 1A, and a cross-sectional view taken along the line 1-1 in FIG. 1A is shown in FIG. 1B. FIG. 2 shows a block diagram of the thermometer pad 1. The thermometer pad 1 is preferably used in combination with a reader 2 in a temperature measuring system shown in FIG. 3.

As shown in FIG. 3, the adhesive clinical thermometer pad 1 is preferably attached to a skin surface of a patient for measuring the body temperature. This thermometer pad 1 is preferably used in combination with a reader 2 for reading the temperature information from the thermometer pad 1 and storing the temperature information therein. The reader 2 is preferably configured so as to be detachably plugged into a socket 3 having a charge function to be connected to the personal computer 4 via a cable 5 so that data processing can be performed by the personal computer 4.

In this embodiment, preferably the clinical thermometer pad 1 attached to, e.g., a skin surface of a patient P receives a radio wave R1 emitted from the reader 2 and generates electric power from the received radio wave R1 by itself, and measures the body temperature using the self-generated electric power. The measured temperature data will be transmitted as a radio wave R2 from the clinical thermometer pad 1 to the reader 2 together with a given identification code (hereinafter referred to as “ID code”) of the clinical thermometer pad 1, and then stored in the reader 2. After completing the temperature measurement, the reader 2 is preferably plugged into the socket 3 to be connected to the personal computer 4 via the cable 5. The personal computer 4 reads the temperature information from the reader 2 and can perform various data processing depending on need.

FIG. 2 shows a block diagram of an illustrative embodiment of the adhesive clinical thermometer pad 1. As shown in FIG. 2, the adhesive clinical thermometer pad 1 preferably includes, e.g., an IC chip 6, a temperature sensor (e.g., a thermistor) 25, an antenna 22, an external interface 1F 26 and an external temperature sensor (e.g., a thermistor) 30. The aforementioned IC chip 6 preferably includes, e.g., a CPU 20, an EEPROM 23 storing an ID code of the adhesive clinical thermometer pad 1 and programs, an A/D converter (hereinafter simply referred to as “A/D”), an RF portion 21 and a power generation circuit 27 for generating electric power by rectifying the RF carriers of the radio wave received by the antenna 22.

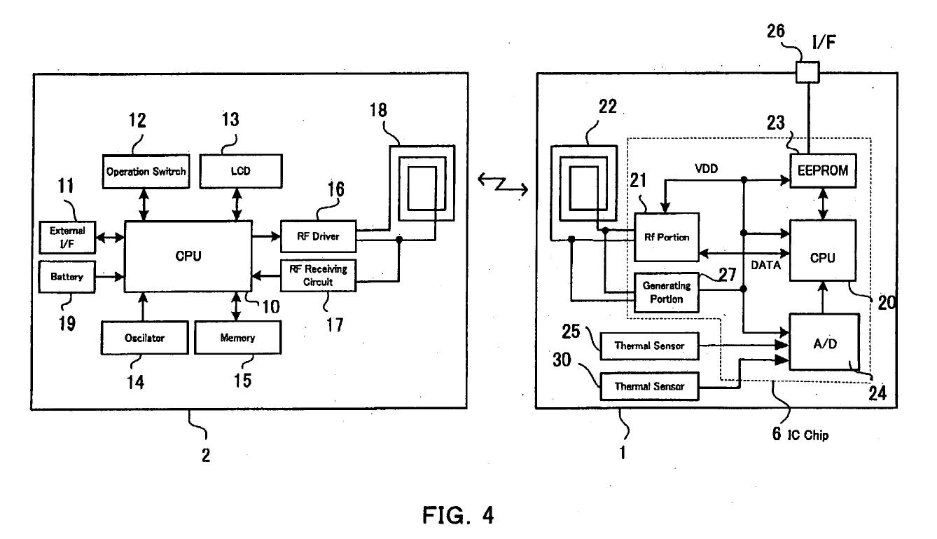

FIG. 4 shows a block diagram of an illustrative entire system including the aforementioned block diagram of the adhesive clinical thermometer pad 1. As shown in the left side block diagram in FIG. 4, the reader 2 preferably includes, e.g., a processor 10 (hereinafter referred to as “CPU”) for, e.g., entirely controlling the reader 2, an external interface 11 (hereinafter referred to as “I/F”) for, e.g., exchanging data between the reader 2 and an external personal computer 4, an operation switch 12 for, e.g., operating the reader 2, a liquid crystal display (LCD) 13, an oscillator 14 for a system clock and a clock function, a memory 15 for temporarily storing received data, an RF driver 16 including a resonant circuit, an RF receiving circuit 17, and antenna 18 for, e.g., emitting a radio wave and receiving the temperature information from the adhesive clinical thermometer pad 1.

In measuring the body temperature using the aforementioned system, as shown in FIG. 3, the reader 2 is preferably unplugged from the socket 3 by which the reader 2 was being charged. Then, the reader 2 is preferably brought close to the adhesive clinical thermometer pad 1 attached to the skin surface of a patient R In this state, when the operation switch 12 is turned on, the reader 2 emits a 13.56 MHz weak radio wave R1 in the order of approximately 10 mW via the antenna 18 toward the adhesive clinical thermometer pad 1. The adhesive clinical thermometer pad 1 adhering to the skin surface of the patient P receives the radio wave and rectifies the RF carriers of the radio wave R1 to thereby generate electric power. The IC chip 6 embedded in the pad 1 capable of, e.g., being operated by the generated electric power measures the body temperature with the temperature sensor 25.

Preferably, the measured body temperature data is wirelessly transmitted with, e.g., a radio wave R2 together with the ID data of the adhesive clinical thermometer pad 1 stored in the EEPROM 23 in the IC chip 6 via the RF portion 21 and the antenna 22.

The reader 2 preferably receives the radio wave R2 including the body temperature data wirelessly transmitted from the antenna 22 of the adhesive clinical thermometer pad 1, and then converts the temperature data into digital data. The digitalized data of the body temperature information can be stored in the memory 15 with the time data related to the body temperature data. The reader 2 can have an alarm function that discriminates whether the body temperature exceeds a predetermined temperature and sounds an alarm when it is discriminated that the body temperature exceeds the predetermined temperature.

When the reader 2 is, e.g., plugged into the socket 3 connected to the personal computer 4 via the cable 5, the information including the body temperature and the ID code of the pad 1 and the measured date and time can be transmitted to the personal computer 4 via the cable 5, and then stored in a hard disk HDD (not shown). Thus, in such a manner, a series of operations for measuring body temperature, recording the body temperature and storing the temperature information may be completed.

In various examples, the temperature sensor 25 can be any means capable of converting a detected temperature into an electric resistance. Examples thereof include a thermistor chip and a thermistor pattern printed on a film-like substrate. Preferably, the temperature sensor 25 embedded in the measuring pad 1 directly or indirectly adheres to the skin surface of the patient P for a long time period. Accordingly, the actual and accurate body temperature can be quickly measured without requiring any prediction time which is usually required in a normal prediction type clinical thermometer. This remarkably reduces measurement errors.

As shown in FIG. 1, the adhesive clinical thermometer pad 1 is preferably formed into a generally round disk shape. The main body 1a is preferably made with, for example, a polyurethane foam. The bottom surface in the central portion of the main body 1a is preferably provided with a dented portion 1b having a certain depth. In the bottom of this dented portion 1b, the thermistor chip 25 (e.g., a temperature sensor) is disposed so that the thermistor chip 25 can be isolated from the outside air. This thermistor chip 25 can, thus, detect indirectly the body temperature of the patient in the state in which the pad 1 adheres to a skin surface of a patient. Since the thermistor chip 25 is thermally insulated from the external air, it becomes possible to measure the body temperature more accurately.

Preferably, also embedded in the main body 1a are an antenna 22 and the IC chip 6. The antenna 22 is formed into, e.g., a generally circular shape along the periphery of the main body 1a. The shape and the structure of the antenna 22 are not limited to the above, and can be any shape and structure. In the preferred embodiments, the pad 1 is further provided with an additional thermistor 30 for measuring an external temperature. This additional thermistor 30 is preferably arranged at the upper surface side of the main body 1a so as to be exposed to the external air. By considering the external temperature measured with this thermistor 30, the body temperature measured with the thermistor chip 25 can be amended so as to obtain accurate body temperatures of the patient. On the bottom surface of the main pad 1a, an adhesive layer 1c is preferably formed so that the entire pad 1 can immovably adhere to a skin surface of a patient. In place of forming the aforementioned adhesive layer 1c, an adhesive tape (not shown) can be provided on the bottom surface of the main body 1a. Alternatively, any other means for adhering or attaching the pad 1 to, e.g., a skin surface of a patient can be employed.

In the above explained embodiment, although the adhesive clinical thermometer pad 1 is formed into a round shape with a relatively large thickness, the structure of the adhesive clinical thermometer pad 1 is not limited to the above. In place of the above, the structure disclosed in PCT/JP03/03437 and Unexamined Japanese Laid-open Patent Publication No. 2003-270051 can also be employed, and the disclosures thereof are incorporated herein by reference in their entireties, such incorporation being not merely in relation to the pad structure, but in relation to each and every aspect of such disclosures.

It should be understood that in this disclosure the wording of “pad” does not always mean a “relatively thick cushion-like member made of soft material” as shown in FIG. 1, but also means any other various members such as a sheet-like member, a film-like member, a patch-like member, a plate-like member or a belt-like member. Among other things, it is preferable that the clinical thermometer pad 1 is a soft and flexible flattened member capable of fitting to a skin surface of a human body along the curvature thereof.

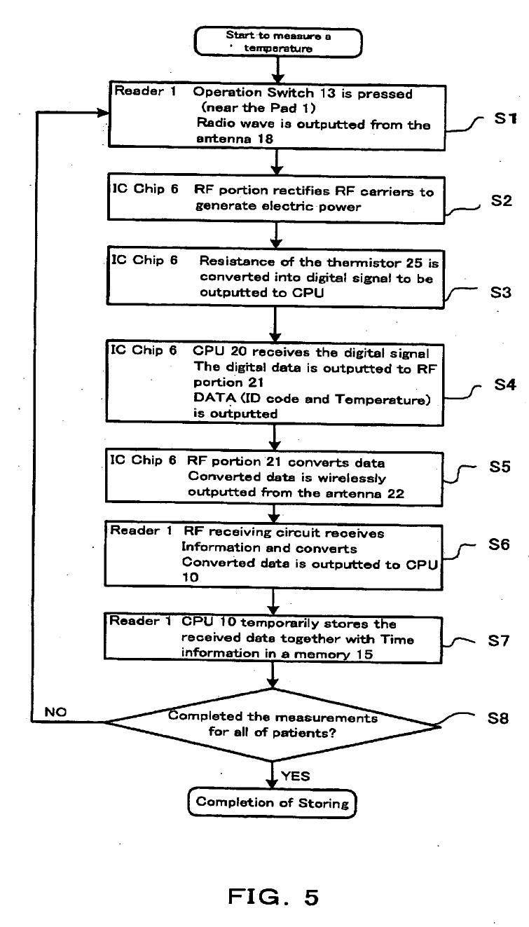

The preferred operation of this illustrative temperature measuring system will be explained based on the flowchart shown in FIG. 5. In this disclosure, “Step” may be simply referred to as “S.”

Initially, the operation switch 12 of the reader 2 is preferably turned on near the adhesive clinical thermometer pad 1 to output a weak radio wave in the order of 10 mW generated in the RF driver 16 from the antenna 18 (Step S1).

Preferably, the radio wave is received by the antenna 22 of the adhesive clinical thermometer pad 1 and introduced into the RF portion 21 of the IC chip 6. The RF portion 21 rectifies the RF carrier of the radio wave to generate the electric power, i.e., power-supply voltage VDD, which is supplied to the entire portion of the IC chip 6 (Step S2).

Preferably, the temperature sensor 25, or a thermistor 25 which varies in electric resistance in accordance with the body temperature of a human body, converts the electric resistance thereof into a voltage. The voltage is applied to the A/D converter 24 in which the voltage is converted into digital data, and then the digital data is outputted to the CPU 20 (Step S3).

The CPU 20 receives the digital data and makes a register store the data. The CPU 20 outputs digital data temporarily stored in the register to the RF portion 21 with the ID code previously written in the EEPROM 23 associated with the digital data (Step S4).

Preferably, the RF portion 21 converts the digital data into a wireless temperature data and then wirelessly outputs the temperature data via the antenna 22 (Step S5).

On the other hand, in the reader 2, the RF receiving circuit 17 preferably wirelessly receives the temperature data from the pad 1 via the antenna 18 and then converts the data Into digitalized temperature data and outputs the data to the CPU 10 (Step S6).

The CPU 10 makes the memory 15 store the digitalized temperature data together with the current time information (Step S7).

Thus, the processing from the measurement of body temperature to the recordation of temperature information for a single person (e.g., patient) is completed. Then, it is discriminated whether processing for all persons (e.g., patients) is completed (Step S8).

If it is discriminated that processing for all persons (e.g., patients) is completed, the job terminates. To the contrary, if it is discriminated that processing for all persons (e.g., patients) is not completed, the routine returns to Step S1 to repeat the aforementioned steps from Step S1 to Step S8,

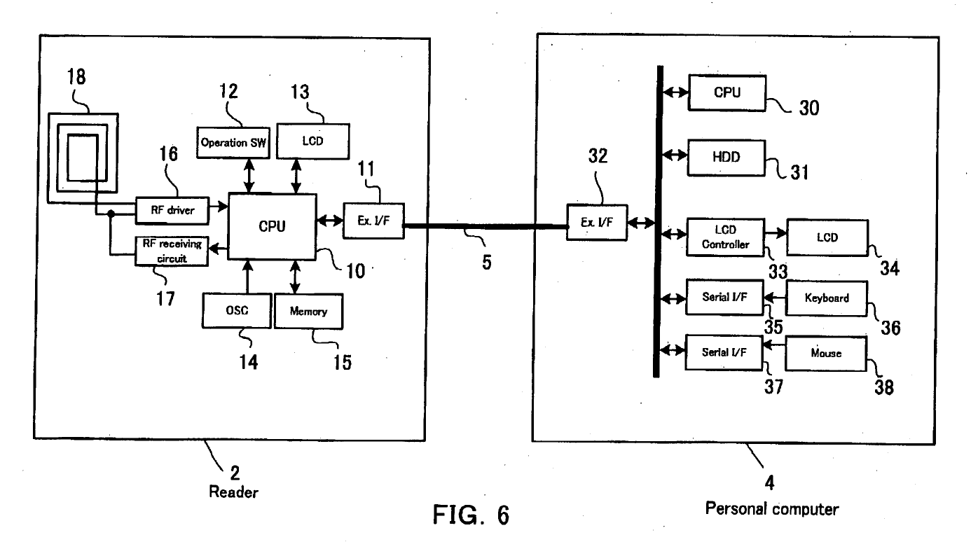

FIG. 6 shows a block diagram of the reader 2 and that of the computer 4 connected thereto via, e.g., a wire 5 in a state in which the reader 2 is plugged into the socket 3. Since the block diagram of the reader 2 is preferably substantially the same as that of the reader shown in FIG. 4, the explanation will be omitted by allotting the same reference numerals to the corresponding portions. In the right side block diagram showing the computer 4, reference numeral “30” denotes a CPU capable of executing an operation system (hereinafter referred to as “OS”), “31” denotes a hard disk (hereinafter referred to as “HDD”) capable of storing various application software and the data from the reader 2, “32” denotes an external I/F such as a USB port connected to the internal bus, “33” denotes an LCD controller, “34” denotes an LCD monitor, “35” denotes a serial I/F, “36” denotes a key board connected to the serial I/F 35, “37” denotes a serial I/F, “38” denotes a mouse connected to the serial I/F 37.

When the reader 2 is plugged into the socket 3 after the completion of measurements for all of the patients, the data stored in the memory 15 is transmitted from the external I/F 11 to be transferred to the personal computer 4 via the cable 5. In the personal computer 4, the data is received by the external I/F 32 and then transferred to the HDD 31. This HDD 31 stores the data (including, e.g., data of the ID of each patient, the body temperature, and the measured time and date).

In this embodiment, the data transfer from the reader 3 to the computer 4 is performed via the cable 5 (i.e., a cable communication). In place of such a cable communication for the data transfer, another method, such as, e.g., a known wireless communication method can be employed.

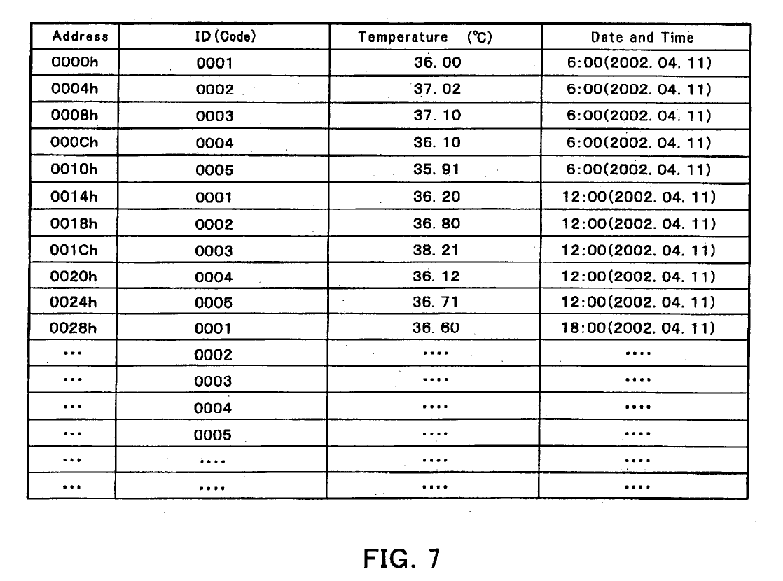

FIG. 7 shows the temperature information data stored in the HDD 31. The data can be, e.g., contained within a database including the data of the ID code, the measured body temperature and the measured time and date stored in this order for every patient. The data contained in this database can be utilized using application software capable of being operated by the CPU 30.

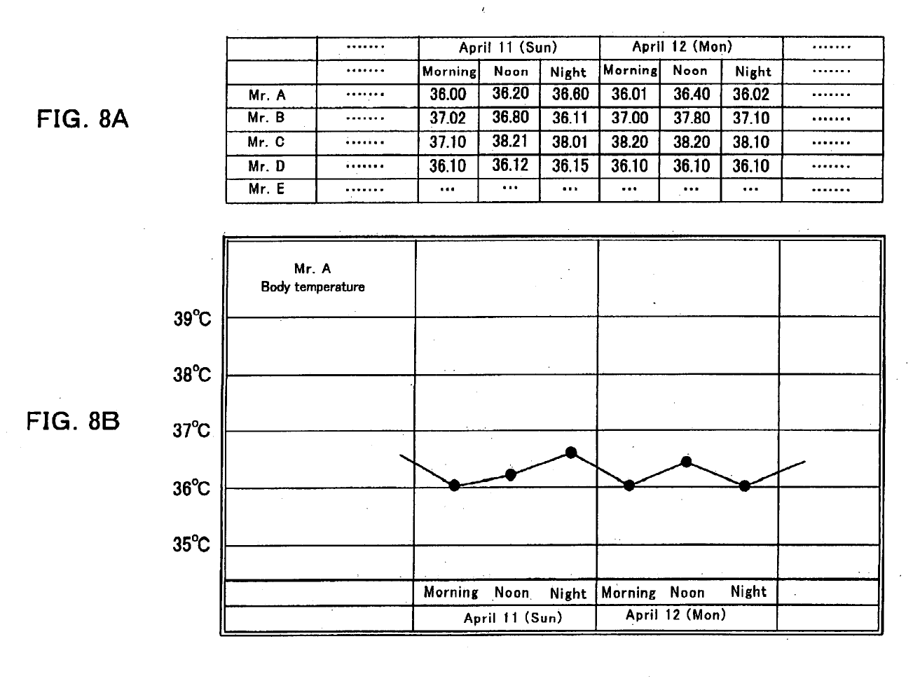

An illustrative example of utilizing the database is shown in FIGS. 8A-8B. In this regard, FIG. 8A shows a table displayed on the monitor of the computer 4 in which, by way of example, a two-day-history of the body temperatures of each patient measured three times a day is displayed. This history can also or alternatively be displayed as a graph shown in FIG. 8B, for example. The graph can be displayed, e.g., as a unit such as a one-day-history, a three-day-history, or a one-week-history, which is useful for a nurse or other caretaker to easily and visually grasp the status of each patient.

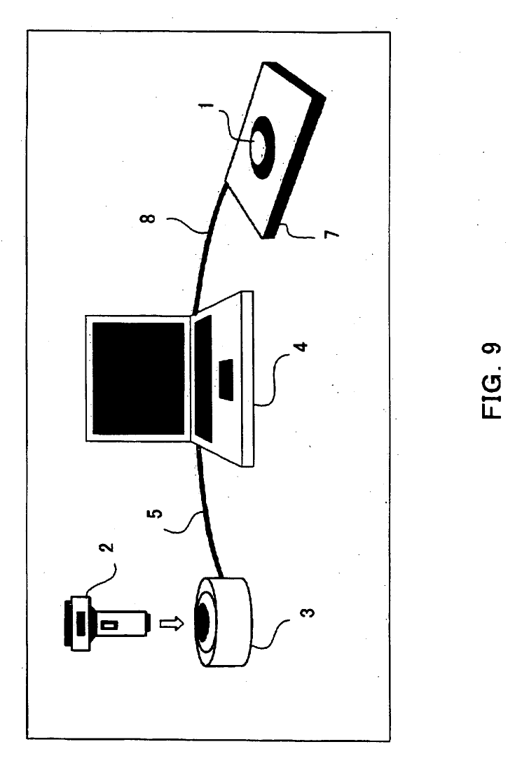

As mentioned above, the aforementioned adhesive clinical thermometer pad 1 preferably stores the ID code given to each pad 1 which is also preferably exclusively used for a certain patient. Therefore, each ID code preferably corresponds to a respective patient. In cases where the pad 1 is used by another patient, the ID code should preferably be changed. Accordingly, in some embodiments, as shown in FIG. 9, the system further includes, e.g., an ID rewriting table 7 for rewriting the ID code of each pad 1 stored in the EEPROM 23. The rewriting table 7 is connected to the personal computer 4 via a cable 8.

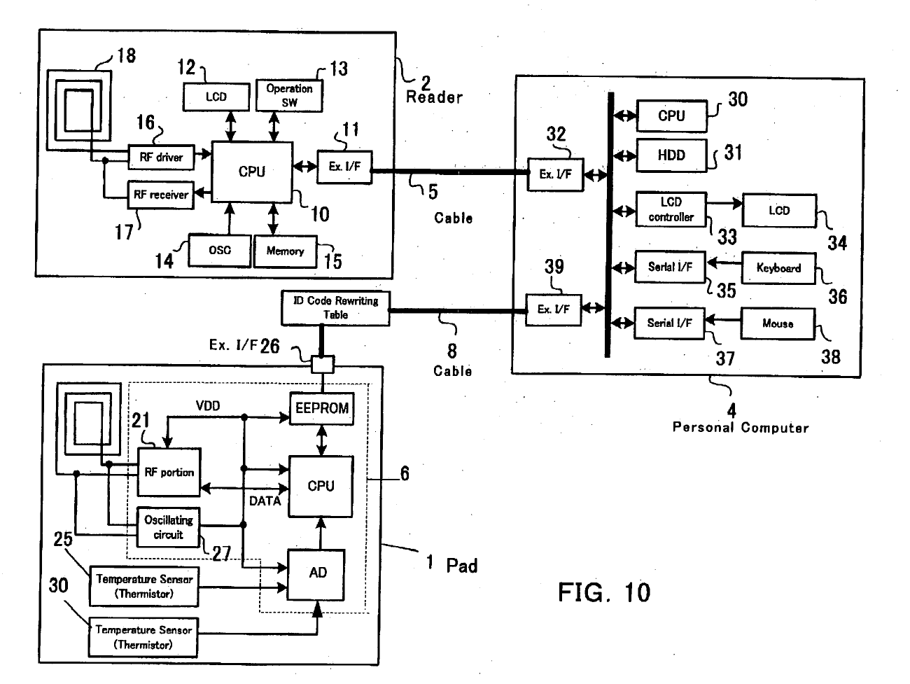

In rewriting the ID code of the pad 1, the pad 1 can be disposed on the table 7 with the external I/F 26 of the pad 1 connected to the table 7, and then the rewriting table 7 is preferably operated by the personal computer 4. Thus, the ID code stored in the EEPROM 23 of the pad 1 can be easily rewritten. The block diagram showing the connected status is shown in FIG. 10. Since the structures thereof are preferably substantially the same as that those shown in FIG. 1, a detailed explanation of this block diagram will be omitted by allotting the same reference numerals to the corresponding portions.

In the aforementioned embodiment, the electric power for driving the IC chip 6 of the pad 1 is preferably generated by rectifying the RF carriers of the radio wave emitted from the reader 6 and received by the pad 1. In other embodiments of the present invention, however, another power source, such as, e.g., a battery (not shown), can be used for driving the IC chip 6.

Furthermore, although the temperature measuring system in the aforementioned embodiment is used for the clinical purposes in hospitals, the system can also be applied to various fields for measuring a temperature, such as, e.g., for measuring a surface temperature of an object.

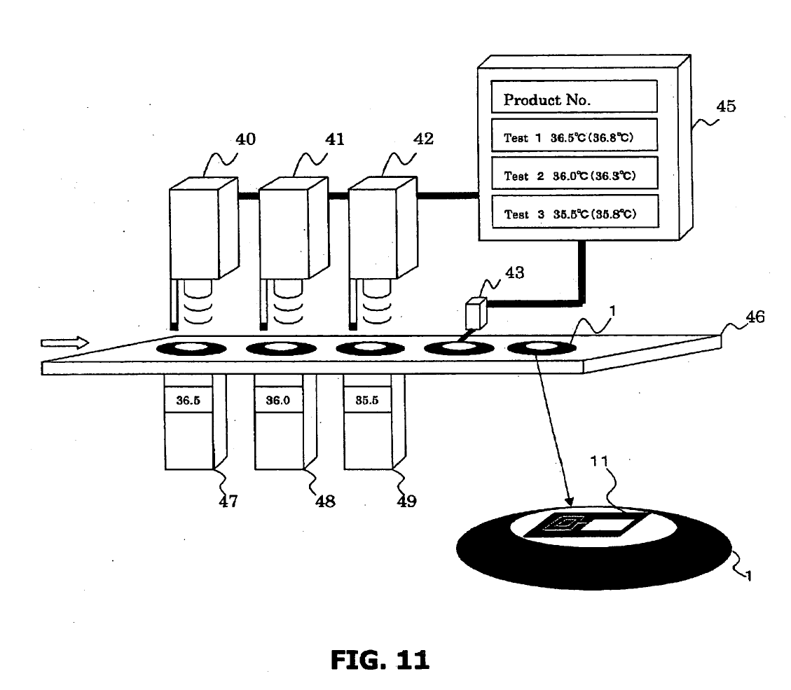

As explained above, the above mentioned temperature measuring pad 1 is provided with a thermistor 25 as a temperature temperature sensor. In manufacturing such thermistors, thermistors different in characteristic can be obtained, which may affect accuracy of measuring temperature. Furthermore, even if thermistors equal in characteristic can be manufactured, it was difficult to manufacture attachable thermometers with a built-in thermistor such that all of the thermometers have temperature measuring accuracy falling within a predetermined accuracy range due to assembling variations and other factors. Accordingly, tight selection tests and/or processing will be required. This may cause a large number of defective goods, which in turn increases the manufacturing cost. Under the circumstances, in the preferable embodiment the temperature data obtained by the thermistor 25 will be corrected. The detail explanation will be made with reference to FIGS. 11 to 17.

In FIG. 11, reference numeral “1” denotes the thermometer pad, “40, 41, 42” denote a temperature reader with a thermometer, respectively, which is capable of transmitting a radio wave toward the pad and receiving a radio wave including temperature information from the pad in the same manner as in the aforementioned reader 2. Reference numeral “43” denotes a writer for writing data into the EEPROM 23 via the external I/F 26, “45” denotes a temperature displaying device for displaying a test temperature and an actually measured temperature, “46” denotes a belt conveyor on which thermometer pads 40, 41 and 42 are to be disposed, and “47, 48 and 49” denote a heater for heating the thermometer pad 40, 41 and 42 via the belt conveyer 46 so as to be a predetermined temperature.

After completing the manufacturing process, as shown in FIG. 11, thermometer pads 1 are disposed on the belt conveyer 46 for conducting a first to third temperature measuring tests 1, 2 and 3 using the temperature readers 40, 41 and 42 and the heaters 47, 48 and 49.

In the first temperature measuring test 1, the thermometer pad 1 is heated to a temperature of 36.5° C. with the heater 47 and a radio wave is emitted from the temperature reader 40. Then, the clinical thermometer pad 1 disposed on the belt conveyer 46 receives the radio wave emitted from the temperature reader 40 and generates electric power from the received radio wave by itself, and measures the temperature using the self-generated electric power. The measured temperature data will be transmitted as a radio wave from the thermometer pad 1 to the temperature reader 40.

Provided that all of the manufactured thermal sensors 25, i.e., thermistors, built-in the thermometer pads 1 are equal in characteristic and the assembling can be performed equally among the thermometer pads 1, the actually measured temperature included in the measured temperature data transmitted from the thermometer pad 1 via the antenna 22 will be 36.5° C. However, the actually measured temperature will not always be 36.5° C., but may be a different temperature such as, e.g., 36.8° C. This means that the thermometer pad 1 recognized the test temperature as a temperature higher than the test temperature of 36.5° C. by 0.3° C. In other words, this tested thermometer pad 1 includes a measuring error in accuracy of temperature measuring.

After the completion of the first temperature measuring test 1 of the thermometer pad 1, the belt conveyer 46 will be advanced toward the right hand side in FIG. 11 for a certain distance, and the second temperature measuring test 2 is performed using the temperature reader 41 and the heater 48 in the same manner as in the first temperature measuring test 1. In detail, in the second temperature measuring test 2, the thermometer pad 1 is heated to a temperature of 36.0° C. with the heater 48 and a radio wave is emitted from the temperature reader 41. Then, the clinical thermometer pad 1 receives the radio wave emitted from the temperature reader 41 and generates electric power from the received radio wave by itself, and measures the temperature using the self-generated electric power The measured temperature data will be transmitted as a radio wave from the thermometer pad 1 to the temperature reader 41. In the second temperature test 2, it is assumed that the measured temperature was 36.3° C.

In the same manner as in the first and second temperature tests 1 and 2, a third temperature test 3 will be performed using the temperature reader 42 and the heater 49. In the third temperature test 3, it is assumed that the measured temperature was 35.8° C.

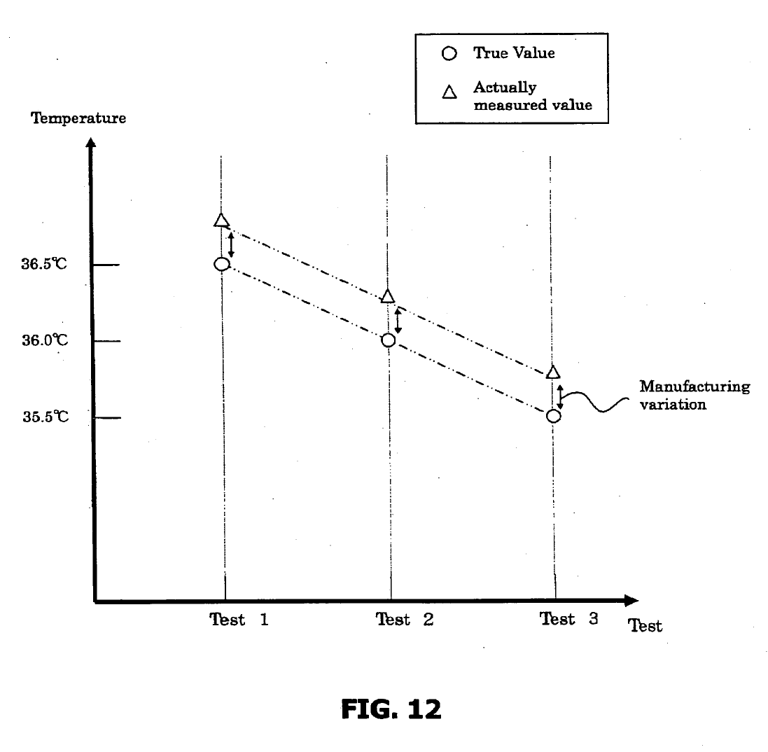

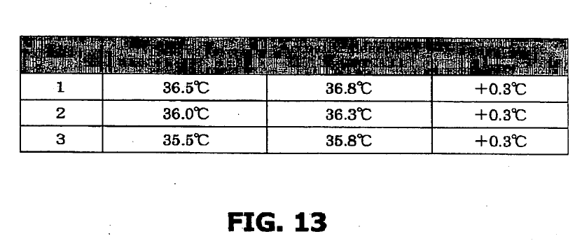

An example of test results of the first to third temperature measuring tests 1 to 3 is shown in FIGS. 12 and 13.

The temperature used in each temperature measuring test as a reference is an ideal value, i.e., an expected value. On the other hand, the measured temperature obtained by converting an analog signal from the thermal sensor 25 built-in the thermometer pad 1 into a digital signal with the A/D converter 24 is an actually measured value. From the FIGS. 12 and 13, the correlation between the expected value and the actually measured value can be obtained. In the first to third temperature measuring tests 1 to 3, it is understand that each actually measured temperature was higher than the expected value by +0.3° C. Accordingly, the expected value can be obtained by correcting −0.3° C., i.e., subtracting 0.3° C. from the actually measured temperature. In other words, in this embodiment, the correction value is −0.3° C.

Accordingly, the writer 43 writes the correction value, e.g., −0.3° C. in this embodiment, on the EEPROM 23 built-in the thermometer pad 1 via the external I/F 26. The EEPROM 23 stores the correction value, i.e., −0.3° C. in this embodiment. Thus, in the actual use, the CPU 20 in thermometer pad 1 will correct the measured value, which was obtained by converting an analog signal from the thermal sensor 25 into a digital signal with the A/D converter 24, into the corrected value using the correction value, and then wirelessly transmits the corrected temperature information from the RF portion 21 via the antenna 22.

As explained above, in this embodiment, the correction value was a fixed value, i.e., 0.3° C. However, in place of such a fixed value, a ratio of the actually measured value to the true value can be employed as a correction value. In this case, the actually measured value is corrected by multiplying the ratio, and then temperature information including the corrected temperature value will be wirelessly transmitted from the RF portion 21 via the antenna 22.

Further, in the aforementioned embodiment, the temperature measuring tests 1 to 3 were performed at three different temperature measuring points. However, in the present invention, the number of temperature measuring points is not limited to the above, and can be any increased number. An increased number of temperature measuring points make it possible to obtain a more accurate corrected temperature value from an actually measured temperature.

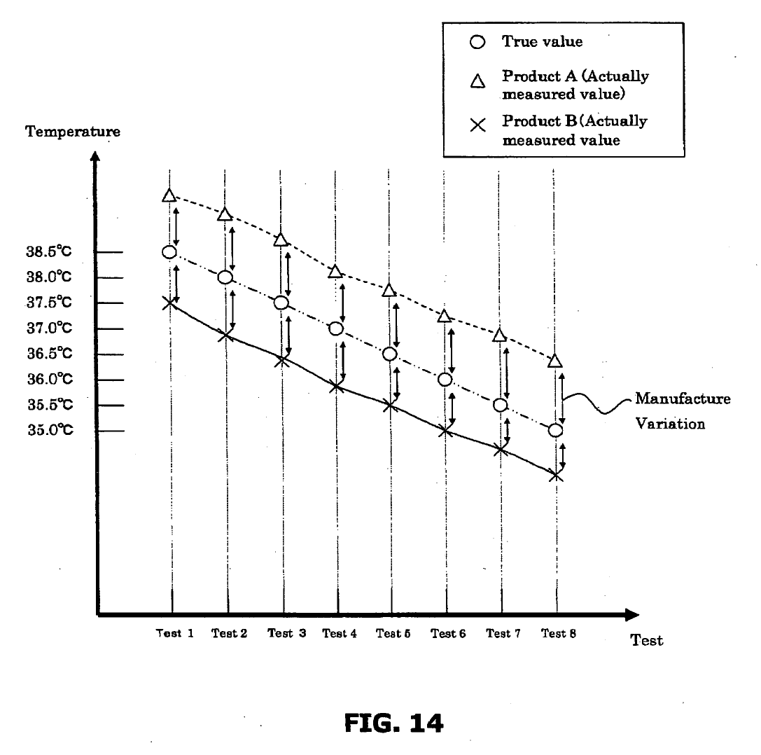

For example, in an embodiment shown in FIG. 14, the temperature measuring tests 1 to 8 were performed at eight different temperature measuring points. In some cases, actually measured values obtained by converting analog signals from the thermal sensor 25 into digital signals with the A/D converter 24 may form a curving line. In such cases, the temperature measuring range can be divided into a plurality of divided ranges, and a correction value or a correction ratio can be changed depending on the divided ranges so that the corrected temperature value approaches the true temperature value.

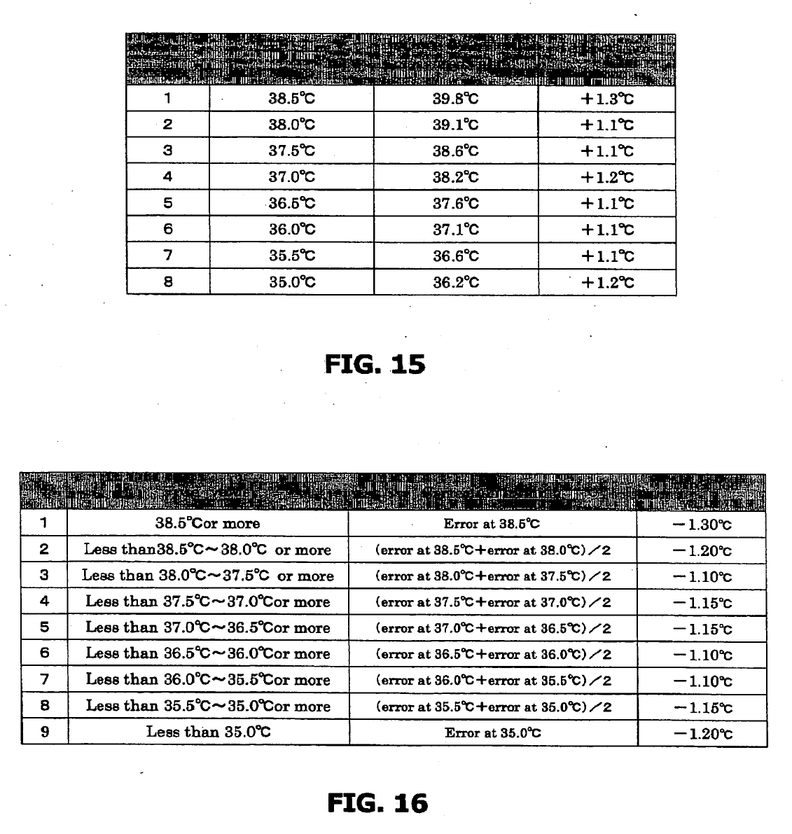

In the case of the product A, as shown in FIG. 15, the temperature errors differ at the temperature measuring test points. In such a case, it is preferable to change the correction value at respective temperature measuring test point.

FIG. 16 shows an example of correction values different at divided temperature ranges. A thermistor used as a thermal sensor does not always have a linear characteristic. In cases where a thermistor 25 built-in the thermometer pad 1 has a non-linear temperature error, it is preferable to divide a temperature measuring range into smaller divided temperature measuring ranges with different correction values. In this example, the correction values were almost constant regardless of the divided temperature measuring ranges. However, in cases where the correction values are not constant, the correction value can be expressed by a mathematical formula.

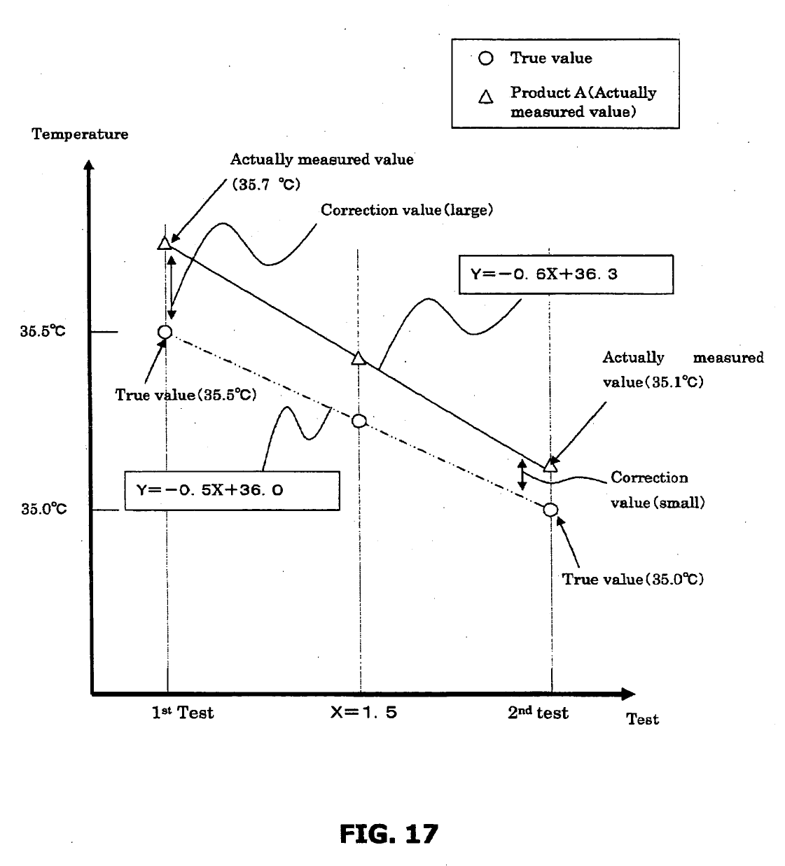

As shown in FIG. 17, in cases where the correction values change at temperature measuring points, a mathematical formula for calculating a true temperature at different temperature measuring points can be obtained. A method for correcting the measured temperatures using a mathematical formula will be explained as follows.

For example, in cases where reference temperatures are 35.5° C. and 35.0° C., a mathematical formula of the linear line connecting the actually measured values of 35.5° C. and 35.0° C. Is obtained. This formula can be easily obtained from concrete two points. If the actually measured temperature value was 35.1° C. when the true temperature value was 35.0° C., the formula of the line connecting the actually measured temperature values will be Y=−0.6X+36.3 as shown in FIG. 17.

From this formula, if the actually measured temperature value Y is known, X can be obtained. Once the value X is obtained, the value X will be substituted for the value X of the formula of Y=−0.5X+36.0 obtained by connecting the true temperature values to thereby obtain the value Y. The calculated value Y will be a temperature data corrected from the actually measured temperature value. For example, if the actually measured value was 35.4° C., X can be obtained from the formula of Y=−0.6X+36.3. That is, X will be 1.5. Then, this value, i.e., X=1.5, is substituted for the X of the formula of Y=−0.5x+36.0 obtained by connecting the true values. As a result, Y=35.25 can be obtained. The calculated value denotes the corrected temperature of 35.25° C.

As will be understood from the above, in cases where correction is made by using a formula, correction values differ at every temperature measuring points, resulting in decreased error. In this example, the formula obtained by connecting the actually measured temperature values expresses a linear line. However, by increasing the temperature measuring points, a formula expressing a curved line can be employed.

In the aforementioned preferable embodiments, the correction of the actually measured temperature is performed by the CPU 20 in the thermometer pad 1. This CPU 20 performs complicated processing to obtain correction values. This may sometimes increase the circuit size and/or power consumption. Accordingly, in order to decrease the size and low power consumption of the thermometer pad 1, the calculation processing can be performed outside the thermometer pad 1. For example, in some preferred embodiments, no correction processing of actually measured temperature values is performed by the CPU 20 in the thermometer pad 1, and data including the actually measured temperature value and the correction value stored in the EEPROM 23 are transmitted from the RF portion 21 via the antenna 22. Thereafter, in the external receiving device receives the data and corrects the actually measured data using the correction value.

Concepts, features and specific embodiments of a temperature measuring device and method disclosed in PCT/JP03/03437, filed on Mar. 20, 2003, can also be applied to the adhesive clinical thermometer pad and the temperature measuring pad according to the present invention, and therefore the entire disclosure thereof. Is incorporated herein by reference in its entirety.

While the present invention may be embodied in many different forms, a number of illustrative embodiments are described herein with the understanding that the present disclosure is to be considered as providing examples of the principles of the invention and such examples are not intended to limit the invention to preferred embodiments described herein and/or illustrated herein.

While Illustrative embodiments of the invention have been described herein, the present invention is not limited to the various preferred embodiments described herein, but includes any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to examples described in the present specification or during the prosecution of the application, which examples are to be construed as nonexclusive. For example, in the present disclosure, the term “preferably” is non exclusive and means “preferably, but not limited to.” In this disclosure and during the prosecution of this application, means-plus-function or step-plus-function limitations will only be employed where for a specific claim limitation all of the following conditions are present in that limitation: a) “means for” or “step for” is expressly recited; b) a corresponding function is expressly recited; and c) structure, material or acts that support that structure are not recited. In this disclosure and during the prosecution of this application, the terminology “present invention” or “invention” is meant as a non-specific, general reference and may be used as a reference to one or more aspect within the present disclosure. The language present invention or invention should not be improperly interpreted as an identification of criticality, should not be improperly interpreted as applying across all aspects or embodiments (i.e., it should be understood that the present invention has a number of aspects and embodiments), and should not be improperly interpreted as limiting the scope of the application or claims. In this disclosure and during the prosecution of this application, the terminology “embodiment” can be used to describe any aspect, feature, process or step, any combination thereof, and/or any portion thereof, etc. In some examples, various embodiments may include overlapping features. In this disclosure and during the prosecution of this case, the following abbreviated terminology may be employed: “e.g.” which means “for example;” and “NB” which means “note well.”

1. A thermometer, comprising:
a thermal sensor that changes in resistance in response to temperature changes;
a memory; and
a temperature correction circuit,
wherein the memory stores, in advance, a temperature difference between an expected measured temperature as a true temperature and an actually measured temperature corresponding to the expected measured temperature obtained by converting an analog signal from the thermal sensor into a digital signal as a correction value, and
wherein the temperature correction circuit corrects a temperature measured in an actual use with the correction value.

2. The thermometer as recited in claim 1, wherein the thermal sensor is a thermistor.

3. A thermometer to be used in combination with a reader for reading temperature information from the thermometer, the thermometer, comprising:
an antenna portion for receiving a radio wave emitted from a reader;
an electric power generating portion for generating electric power with the radio wave received by the antenna portion;
a thermal sensor that changes in resistance in response to temperature changes;
an A/D converter for converting an analog signal from the thermal sensor into a digital signal;
an output portion for wirelessly outputting temperature information toward the reader;
a memory; and
a temperature correction circuit,
wherein the memory stores, in advance, a temperature difference between an expected measured temperature as a true temperature and an actually measured temperature corresponding to the expected measured temperature obtained by converting an analog signal from the thermal sensor into a digital signal as a correction value,
wherein the temperature correction circuit corrects a temperature measured in an actual use with the correction value to obtain a corrected temperature, and
wherein temperature information including the corrected temperature is wirelessly outputted toward the reader from the output portion via the antenna portion.

4. The thermometer as recited in claim 3, wherein the thermal sensor is a thermistor.

5. A thermometer to be used in combination with a reader for reading temperature information from the thermometer, the thermometer, comprising:
an antenna portion for receiving a radio wave emitted from a reader;
an electric power generating portion for generating electric power with the radio wave received by the antenna portion;
a thermal sensor that changes in resistance in response to temperature changes;
an A/D converter for converting an analog signal from the thermal sensor into a digital signal;
an output portion for wirelessly outputting temperature information toward the reader;
a memory; and
a temperature correction circuit,
wherein the memory stores, in advance, at least one expected measured temperature as a true temperature and at least one actually measured temperature corresponding to the at least one expected measured temperature obtained by converting the analog signal from the thermal sensor into the digital signal,
wherein the temperature correction circuit corrects a temperature measured in an actual use with a ratio of the at least one actually measured temperature to the at least one expected measured temperature as a true temperature to obtain a corrected temperature, and
wherein temperature information including the corrected temperature is wirelessly outputted toward the reader from the output portion via the antenna portion.

6. The thermometer as recited in claim 5, wherein the thermal sensor is a thermistor.

7. A thermometer to be used in combination with a reader for reading temperature information from the thermometer, the thermometer, comprising:
an antenna portion for receiving a radio wave emitted from a reader;
an electric power generating portion for generating electric power with the radio wave received by the antenna portion;
a thermal sensor that changes in resistance in response to temperature changes;
an A/D converter for converting an analog signal from the thermal sensor into a digital signal;
an output portion for wirelessly outputting temperature information toward the reader;
a memory; and
a temperature correction circuit,
wherein the memory stores, in advance, a plurality of expected measured temperatures as true temperatures and a plurality of actually measured temperatures each corresponding to each of the plurality of expected measured temperatures obtained by converting the analog signal from the thermal sensor into the digital signal,
wherein the temperature correction circuit corrects a temperature measured in an actual use with a temperature difference between the expected measured temperature and the actually measured temperature corresponding to the temperature measured in an actual use to obtain a corrected temperature, and
wherein temperature information including the corrected temperature is wirelessly outputted toward the reader from the output portion via the antenna portion.

8. The thermometer as recited in claim 7, wherein the thermal sensor is a thermistor.

9. The thermometer as recited in claim 7, wherein the temperature correction circuit changes the temperature difference as a correction value depending on a divided temperature range corresponding to the temperature measured in an actual use.

10. The thermometer as recited in claim 7, wherein the temperature correction circuit changes the temperature difference as a correction value depending on the temperature measured in an actual use using a mathematical formula.

11. A thermometer to be used in combination with a reader for reading temperature information from the thermometer, the thermometer, comprising:
an antenna portion for receiving a radio wave emitted from a reader;
an electric power generating portion for generating electric power with the radio wave received by the antenna portion;
a thermal sensor that changes in resistance in response to temperature changes;
an A/D converter for converting an analog signal from the thermal sensor into a digital signal;
an output portion for wirelessly outputting temperature information toward the reader;
a memory;
a temperature correction circuit; and
a control circuit,
wherein the memory stores, in advance, temperature differences between a plurality of expected measured temperatures as true temperatures and a plurality of actually measured temperatures corresponding to each of the plurality of the expected measured temperatures each obtained by converting an analog signal from the thermal sensor into a digital signal as a correction value, and
wherein the control circuit controls the output portion so that the output portion transmits the temperature measured in an actual use and the correction value corresponding to the temperature measured in an actual use toward the reader from the output portion via the antenna portion.

12. The thermometer as recited in claim 1, wherein the thermometer is an adhesive thermometer pad used for measuring a body temperature of a patient for clinical purposes.

13. The thermometer as recited in claim 3, wherein the thermometer is an adhesive thermometer pad used for measuring a body temperature of a patient for clinical purposes.

14. The thermometer as recited in claim 5, wherein the thermometer is an adhesive thermometer pad used for measuring a body temperature of a patient for clinical purposes.

15. The thermometer as recited in claim 7, wherein the thermometer is an adhesive thermometer pad used for measuring a body temperature of a patient for clinical purposes.

16. The thermometer as recited in claim 11, wherein the thermometer is an adhesive thermometer pad used for measuring a body temperature of a patient for clinical purposes.
